(19) United States
(12) Patent Application Publication
(10) Pub. No.: US 2026/0076986 A1
(43) Pub. Date: Mar. 19, 2026

(54) A COMBINATION OF SCYLLO-INOSITOL AND FLAVONES

(71) Applicant: EIRGEN PHARMA, LTD., Waterford (IE)

(72) Inventors: Phillip FROST, Miami Beach, FL (US); Antonio CRUZ, Miami, FL (US); John K. CHOE, Mississauga, Ontario (CA); Jane H. HSIAO, Davie, FL (US)

(21) Appl. No.: 19/109,142

(22) PCT Filed: Sep. 1, 2023
(86) PCT No.: PCT/US2023/031914

§ 371 Date: Mar. 6, 2025

Related U.S. Application Data
(60) Provisional application No. 63/404,537, filed on Sep. 7, 2022.
(60) Provisional application No. 63/441,732, filed on Jan. 27, 2023.
(60) Provisional application No. 63/453,583, filed on Mar. 21, 2023.

(51) Int. Cl. A61K 31/7004; A61K 31/352; A61P 25/28

(52) U.S. Cl. CPC A61K 31/7004; A61K 31/352; A61P 25/28

(57) ABSTRACT

The invention relates to the use of scyllo-inositol alone or in combination with other active ingredients including the flavone apigenin to support cognitive health in a subject that takes the product or combination product. The invention relates to compositions comprising scyllo-inositol alone or in combination with other agents including but not limited to a flavone or other substance that upregulates or prevents the downregulation of aquaporin 4 channels. The compositions, which are inclusive of oral dosage forms such as tablets or capsules, are provided to the subject which is inclusive of humans or other mammals such as dogs or cats or other animals in need of cognitive improvement. The combination product(s) break up amyloid beta aggregates and promote the clearance of amyloid beta fibrils from the glymphatic system.

### CROSS REFERENCE TO RELATED APPLICATIONS

This PCT application claims the benefit of U.S. Provisional Application No. 63/404,537, filed Sep. 7, 2022, U.S. Provisional Application No. 63/441,732, filed Jan. 27, 2023, and U.S. Provisional Application No. 63/453,583, filed Mar. 21, 2023, which are all herein incorporated by reference in their entireties.

### BACKGROUND OF THE INVENTION

Alzheimer's disease (AD) is a neurodegenerative disease or disorder that progresses over time to cause cognitive impairment and multiple symptoms and disabilities that impact the daily lives of subjects afflicted with this disease. The numbers of people living with this disease at its various stages is staggering and is estimated to increase to more than 115 million people worldwide by the year 2050. Effective treatments for the progression and/or treatment of Alzheimer's disease remain elusive despite years of effort and billions of dollars spent on drug development research. Before the recent approvals of monoclonal antibody therapy, only three small molecule drugs have been approved in the United States to treat this disease. These include donepezil, rivastigmine and galantamine-none of which are effective at stopping progression of the disease. The prevalence of Mild Cognitive Impairment (MCI) and Alzheimer's Disease (AD) increases in the aging population. The gradual loss of memory associated with MCI is followed a further deterioration of memory and increasing loss of function. In late MCI stage, patients begin to develop dementia and convert to mild AD. These patients can progress and develop moderate and severe AD. Accumulation of amyloid beta in the brain occurs early and is pre-symptomatic, as amyloid burden in the bran continues to increase, the onset of early and late MCI appears and continues to progress in severity until the patient is diagnosed with AD. It is well established that onset of clinical symptoms such as memory loss begins in early and late MCI and is followed by a more rapid decrease in memory and function as the patient develops through Mild, Moderate and eventually Severe AD. Patients with late stages of Moderate and Severe Alzheimer's Disease demonstrate reduced function requiring caregiver assistance, and potentially, mortality. Although the exact mechanisms triggering AD pathologies are not well elucidated, it is well established that the accumulation of amyloid beta fibrils resulting in the formation of plaques are the hallmark of Alzheimer's disease. Further, the accumulation of amyloid beta fibrils in the brain begins to occur some 10-15 years prior to the development of dementia and associated clinical symptoms. The accumulation of fibrils and plaques increases with aging where 30-40% of the population over the age of 55, and over 70% of MCI patients have amyloid beta burden in the brain. The presence of amyloid beta fibrils and plaques results in increasing neurotoxicity and appearance of inflammation in the brain. The increase in these cumulative pathologies are associated with the appearance of more aggressive disease and loss of memory and function in AD patients.

The aging population is rapidly growing globally resulting in an increase in the number of people predisposed or diagnosed with neurological diseases such as Mild cognitive Impairment (MCI) and Alzheimer's Disease (AD). The onset of these neurodegenerative conditions is characterized by the progression of clinical symptoms from loss of memory to more severe loss of cognition and function. The presence of amyloid beta aggregates and plaques, as well as secondary pathologies such as inflammation and neuronal cell death is a hallmark of AD.

The increase in accumulation of amyloid beta in the aging brain is most likely due to the aggregation and formation of amyloid beta oligomers and fibrils, which become trapped and not easily released into the CSF or digested by cells or enzymatically. Once amyloid beta precursor protein is synthesized and appears on the neuronal cell surface, it is cleaved by alpha and beta secretase, producing free amyloid beta peptide. Ageing, cerebrovascular disease, prior exposure to traumatic brain injury, and sleep disruption are established and emerging risk factors for the development of neurodegenerative conditions, including Alzheimer's disease. In animal models of each, glymphatic function is impaired. Given the role of perivascular glymphatic exchange in amyloid-β and tau clearance, impairment of glymphatic pathway function is now proposed to be important in the development of these conditions. While imaging of glymphatic function using dynamic contrast-enhanced MRI (DCE-MRI) has only recently begun, early studies demonstrate that glymphatic function in humans is impaired in normal-pressure hydrocephalus and in the presence of small vessel disease.

The role of glymphatic impairment in the development of other neurodegenerative diseases has not yet been directly evaluated, but emerging data from studies in human populations suggest a role for aquaporin 4 (“AQP4”) in these conditions. In a post-mortem case series, reduced perivascular AQP4 abundance was observed in the frontal cortex of subjects diagnosed with Alzheimer's disease, while preservation of perivascular AQP4 abundance was observed in subjects remaining cognitively intact over the age of 85. The reduced perivascular AQP4 abundance was further associated with increasing amyloid-β and tau pathology, as well as with global measures of cognitive decline. In three recent genetics studies carried out in distinct human populations, single nucleotide polymorphisms in the human AQP4 gene were associated with variation in cognitive decline, amyloid burden and clinical status, and an association between sleep disruption and amyloid burden. A recent human transcriptomic study further demonstrated that in addition to the expression of AQP4, differences in the expression of genes whose products determine perivascular AQP4 localization (specifically genes encoding elements of the DAC, SNTA1, DINA, IMID, DAG1) were associated with dementia status and temporal cortical tau pathology. These findings suggest that changes in AQP4 expression and localization may contribute to the development and progression of neurodegenerative diseases, including Alzheimer's disease, in human populations. Understanding the emerging role of dynamic AQP4 subcellular relocalization provides a new framework to understand waste clearance in the healthy brain and opens up new treatment avenues to slow the progression of neurodegenerative diseases.

Over the last 20 years, a number of therapies targeting amyloid beta fibrils and plaques have shown promise in animal models for AD and advanced into human clinical trials. These initial therapies involved antibodies reactive with amyloid beta fibrils and plaques resulting in reduced amyloid beta accumulation in brain of AD animal models. Elan and Wyeth developed a humanized antibody, Bapineuzamab, which showed promise in phase 2 studies and advanced into phase 3 clinical trials in mild and moderate AD patients. Unfortunately, the study did not show efficacy in mild to moderate AD patients, similar outcomes were observed with antibody therapies by Lilly and others. The general consensus was that treatment of moderate AD disease with amyloid targeted therapies was too late in the disease process to prevent neurotoxicity and progression of dementia. Some of the evidence that amyloid beta targeted therapies may be more effective in subpopulations of AD patients came from clinical studies showing a modest effect in APOE 4 positive mild AD patients which was masked by the rapid decline in cognition and function observed in the moderate AD patient population.

Further, small oral molecules such as scyllo-inositol and tramiprosate, also showed promising data in disaggregating amyloid beta fibrils in vitro and reducing plaques in AD animal models. Neurochem Pharma initiated an 18 month phase 3 clinical trial in mild to moderate patients. The study failed to show an improvement in cognition and function in AD patients. The data was not conclusive, where the variability in data between patients was very high and it appeared that cognitive symptoms in patients with mild disease did not progress sufficiently during the 18 month treatment period to measure efficacy. There appeared to be two issues arising in these trials, amyloid beta targeted drugs do not appear to impact AD cognition and function in moderate AD patients, indicating that pathology of the disease process is too far advanced to alter the progression with amyloid beta targeted therapies, and secondly that the decline in cognition and function in mild AD patients is very slow over the 18 month treatment period to assess efficacy in the overall Mild AD patient population, more specifically, the reduction in the decline in endpoints that measure cognition and function such as ADASCog, NTB, CDR-SoB, ADCS-ADL and others.

Similarly, scyllo-inositol, an oral agent that disrupts and prevents the formation of amyloid beta fibrils, was effective in treating AD animal models. Following treatment of AD animal models with scyllo-inositol with doses of 3.3 mg/kg or higher resulted in reduced prevalence and size of amyloid plaques, and improvement memory and cognition testing appropriate for the different animal models evaluated. In a large phase 2 trial, scyllo-inositol failed to show efficacy in mild and moderate AD patients in several endpoints, including NTB, CDR-SoB, ADCS-ADL and ADAS-Cog. These data in the overall, treated population in the study indicated that the scyllo-inositol was not necessarily effective in the overall mild and moderate AD population to treat Alzheimer's disease. Further, the data examining mild AD patients with an MMSE from 20-26 or in moderate AD patients with MMSE from 16-20 also showed the drug was not effective for both AD populations under the particular trial conditions to treat Alzheimer's disease. These data suggested that either the drug is not effective for treating mild and moderate AD patients in general or any efficacy occurring on a subpopulation of AD patients was being masked by the data from the overall population. Based on the clinical evidence that amyloid beta targeted drugs may be ineffective in more advanced AD patients, the efficacy of scyllo-inositol was evaluated in mild AD patients with different severities of disease based on MMSE scores from 20-26. The data showed that scyllo-inositol efficacy on cognition and function was more pronounced in mild AD patients with an MMSE score of 22-26. The inclusion of mild AD patients with an MMSE score of 20 and 21 was sufficient to mask the efficacy observed in mild AD patients with an MMSE from 22-26.

These clinical findings highlighted that selecting the appropriate MCI and AD patient population for amyloid beta targeted therapies is essential to observe efficacy. In one embodiment, the present invention comprises treatment of subjects having mild AD with an MMSE score of 22-26 with a pharmaceutically effective amount of scyllo-inositol. The new data evaluated in this subset of patients also suggests that scyllo-inositol is effective in subjects that have mild cognitive impairment and/or memory loss to delay any further loss or mitigate the loss of cognition or memory before or well before the actual onset of the disease. Because of these data, the use of safe dosage amounts of scyllo-inositol alone or in combination with a suitable nutritional or nutraceutical supplement such as apigenin would be particularly beneficial in a younger, healthy population that might be predisposed to developing more severe cognitive or memory disorders in later life.

There is thus a need for an effective nutritional or dietary supplement that comprises scyllo-inositol alone or in a combination product comprising scyllo-inositol and a nutritional or nutraceutical ingredient useful for improving memory or cognitive function in a population of people that may be prone to memory loss or mild cognitive disorders. In a preferred embodiment, the combination is a composition comprising a first component selected from scyllo-inositol and a second component comprising a flavone such as apigenin. There is also a need for a combination product comprising scyllo-inositol and another active ingredient selected from a pharmaceutical product or a supplement or nutraceutical that can be provided to subjects that may be predisposed to early amyloid beta accumulation in the brain that eventually results in progression to MCI, Alzheimer's disease or other cognitive disorders. This need for intervention can arise long before evidence of any neurological symptoms are detected in early stages of MCI and Alzheimer's Disease.

It is believed that with the new discovery of a patient subpopulation having an MMSE score of 22-26 suggests that scyllo-inositol has an immediate impact on improving memory and cognition, which is detectable within the first 6 months of treatment.

It has been postulated that glycolipids such as gangliosides may result in the stabilization and prevention of Ab fibril formation while phosphatidyl inositols may result in the acceleration of fibril formation. Scyllo-inositol (ELND005) has been disclosed as useful in the treatment or prevention of a condition of the central or peripheral nervous system including Alzheimer's disease. See U.S. Pat. No. 7,521,481, which is hereby incorporated by reference. Completed studies in Alzheimer's disease include the study entitled ELND005 in Patients with Mild to Moderate Alzheimer's Disease and Long Term Follow-up Study in Subjects with Alzheimer's disease.

The present inventors have thus discovered a subpopulation of patients within the broader class of Alzheimer's or MCI patients that are effectively treated with a pharmaceutically effective amount of scyllo-inositol alone or in combination with some other active ingredients. It is believed that this subset of patients is comparable to or can be similarly situated to healthy subjects that have the initial stages of fibril formation and who have some sign of memory loss, dementia or mild cognitive disorder.

The use of scyllo-inositol and any of the other active 1,2,3,4,5,6-cycloheanehexols such as cis-, epi-, allo, muco-, neo-, D-chiro and L-chiro-inositols in combination with apigenin is also useful to treat aging patients with memory loss due to increased accumulation of amyloid beta in the brain, optionally having MMSE scores of 22 to 26 and MCI patients predisposed to develop mild to moderate Alzheimer's disease and having MMSE scores of 27 to 28. This drug combination will reduce amyloid accumulation in the brain, as well as reduce the amyloid inhibition of neuronal function. In preferred embodiments, the combinations are fixed dose combinations in the same dosage form such as a tablet or capsule. It is believed such combinations slow the progression in loss of cognition and function in these subgroups of patients having the above MMSE scores and improve memory and cognition or delay the loss of memory or cognition in the healthy population that may be predisposed to accumulating amyloid beta.

The combination treatments also provide more immediate effects on cognition and memory loss or other associated symptoms. The combination may also comprise a combination selected from scyllo-inositol (min of 70 to 500 mg mgs) and apigenin (minimum of 60 to 150 mgs). These amounts can vary depending upon the particular subject. The combination can be in separate doses or in a single dosage form or combination of dosage forms with additional ingredients such as vitamins.

The present invention also includes any other Aquaporin 4 (AQP4X) upregulators inclusive of small molecules or nucleotides such as oligonucleotides that upregulate the expression of AQP4X and that facilitate amyloid beta clearance through this astrocytic water channel. Compounds, which increase read-through of the aquaporin 4 gene, include apigenin, a flavone found in chamomile, and sulphaquinoxaline, an antibiotic (for animals only). Apigenin can also be combined with scyllo-inositol to treat MCI and Alzheimer's patients, in particular those subjects having MMSE scores of 22. Other suitable combination ingredients can include compounds which lower the levels of soluble ST2 (sST2) in the brain, a protein known to negatively impact amyloid clearance from the brain.

The drug combination may be useful as a supplement or a nutraceutical as well depending upon the dosage amount of either a separate combination of scyllo-inositol and another active ingredient such as apigenin.

### SUMMARY OF THE INVENTION

In an embodiment, the present invention comprises a composition comprising a combination of (i) a first compound selected from an inositol compound or pharmaceutically acceptable salts thereof and (ii) a second compound selected from an additional active ingredient wherein the additional active ingredient is selected from the group consisting of an aquaporin 4 (AQP4X) upregulator and relocalization on fibrous astrocytic feet. In an embodiment, the composition is useful to treat a neurological disorder or cognitive deficiency or memory loss in a subject in need of treatment thereof.

In a preferred embodiment, the first compound is selected from scyllo-inositol and the second compound is selected from a flavone such as apigenin or any drug that upregulates the expression of an aquaporin channel. The first compound and the second compounds may be administered separately and provided in discrete dosage forms which are co-administered. The first compound and the second compound may also be packaged as separate dosage forms in the form of a kit that is provided to the patient for daily administration of the first compound and the second compound in suitable dosage forms such as capsules, tablets or ampules. Alternatively, the combination may be in the form of a single dosage form comprising (i) a first compound selected from an inositol and (ii) a second compound selected from an aquaporin 4 upregulator and relocalization. In such single dosage form, the first compound is selected from scyllo-inositol and the second compound is selected from apigenin. In a further preferred embodiment, the dosage form may be in the form of a capsule or tablet or ampule and comprise as a first compound scyllo-inositol and, as a second compound, apigenin.

In those jurisdictions which do not permit medical use claims or method of treatment claims, such embodiments comprise pharmaceutical compositions or combinations of such scyllo-inositol and aquaporin 4 upregulators/relocaliation as combinations or as single unit formulations for use in treating such sub-populations of Alzheimer's or MCI patients or for use in providing healthy subjects with a combination nutritional supplement or novel food substance that is useful as a supplement. Such combinations or compositions are novel and inventive per se. In any of the above uses or as further recited herein, the dosing amounts of scyllo-inositol can also include 70-250 mgs once daily or 35-125 mgs BID.

In an embodiment, the invention comprises a method of reducing brain amyloid beta levels and formation of plaques in healthy subjects comprising the administration of an effective amount of apigenin in combination with an effective amount of scyllo-inositol.

In a further embodiment, the invention comprises a method of treating an Alzheimer's patient having the confirmed presence of amyloid pathology and mild cognitive impairment or mild dementia stage of disease consistent with Stage 3 or Stage 4 Alzheimer's disease comprising the administration of between about 50 to 250 mgs of apigenin and comprising the administration of an effective amount of scyllo-inositol.

In an embodiment, the invention comprises a method of improving memory, cognition and/or brain function in subject comprising co-administration of a nutraceutically effective amount of scyllo-inositol and a nutraceutically effective amount of apigenin wherein such co-administration results in improved memory, cognition and/or brain function relative to a patient being treated with scyllo-inositol or apigenin alone. In such an embodiment, the co-administration may comprise the administration of a composition comprising a fixed-dose combination of scyllo-inositol and apigenin in a single dosage form such as a tablet or capsule. In an embodiment, the combination composition comprising scyllo-inositol and apigenin, in suitable dosage forms, is provided to subjects over the age of fifty at risk for accumulation of amyloid beta. Such subjects may exhibit early signs of memory loss but would benefit from early intervention in a supplement format.

The present invention comprises:
1. A combination of (i) a first compound selected from an inositol or a pharmaceutically acceptable isomer or salt thereof and (ii) a second compound selected from a molecule that upregulates aquaporin 4.
2. The combination according to claim 1 wherein the first compound is selected from scyllo-inositol or other active 1,2,3,4,5,6-cyclohexanehexol and the second compound is selected from the group consisting of molecule flavone.
3. The combination according to claim 2 wherein the first compound is selected from scyllo-inositol and the second compound is selected from apigenin.
4. A composition comprising a flavone and scyllo-inositol.
5. The composition according to claim 4 wherein the composition is a nutraceutical composition comprising apigenin and scyllo-inositol.
6. A dosage form comprising a combination of (i) a first compound selected from an inositol or a pharmaceutically acceptable isomer or salt thereof and (ii) a second compound selected from a flavone.
7. The dosage form according to claim 6 wherein the first compound is selected from scyllo-inositol or other active 1,2,3,4,5,6-cyclohexanehexol and the second compound is selected from apigenin.
8. The dosage form according to claim 7 wherein the first compound is selected from scyllo-inositol.
9. The dosage form according to claim 8 wherein in the form of a solid, oral dosage form.
10. The dosage form according to claim 9 wherein the dose of scyllo-inositol is about 75 to 150 mgs once or twice daily (BID) and the dose of apigenin is about 60-150 mgs per day.
11. The dosage form according to claims 6-10 wherein said dosage form is in the form of a capsule or tablet.
12. The dosage form according to claim 11 in the form of a tablet comprising an immediate release formulation of scyllo-inositol and an immediate release formulation of apigenin.
13. The dosage form according to claim 11 in the form of a tablet comprising an immediate release formulation of scyllo-inositol and an extended release formulation of apigenin.
14. A method of treating a subject having an MMSE score in the range of 22-26 with a pharmaceutically effective amount of scyllo-inositol.
15. The method according to claim 14 wherein the subject has Alzheimer's disease.
16. The method according to claim 14 or claim 15 wherein the pharmaceutically effective amount of scyllo-inositol is about 250 mgs BID per day.
17. A method of treating a subject having a cognitive disorder, memory loss comprising administering an effective amount of scyllo-inositol in combination with a flavone.
18. The method according to claim 17 wherein the flavone is selected from apigenin.
19. A method of providing a subject predisposed to a neurological disease or condition with a nutraceutical supplement comprising obtaining a combination of (i) a first compound selected from an inositol or a pharmaceutically acceptable isomer or salt thereof and (ii) a second compound selected from a flavone and providing said combination to the subject in need thereof.
20. A method of improving memory loss comprising the administration of an oral dosage form comprising scyllo-inositol and apigenin and comprising nutraceutically acceptable excipients.
21. The method according to claim 20 wherein the oral dosage form is in the form of a tablet, which comprises nutraceutically acceptable excipients, selected from at least one of a binder, a filler and a disintegrant.
22. A scored tablet comprising the combination of scyllo-inositol and apigenin and nutraceutically acceptable excipients.
23. Use of a combination of scyllo-inositol in a dosage amount of 75 mgs to 250 mgs once daily or twice daily in combination with a flavone in the manufacture of a nutraceutical ingested by a subject.
24. A combination comprising a first compound selected from scyllo-inositol and a second compound selected from the group consisting of a flavone or other small molecule or an oligonucleotide that modulates the expression of an aquaporin 4 channel.
25. The combination according to claim 24 wherein each compound is in a separate dosage form.
26. The combination according to claim 24 wherein the combination is in a single dosage form.
27. The combination according to claim 24 or 25 wherein the dosage amount of scyllo-inositol is between 70-125 mgs and the dosage amount of apigenin is between 70-125 mgs.
28. The combination according to claim 27 further comprising a vitamin or additional supplement that supports cognitive health.
29. A combination comprising scyllo-inositol and apigenin.
30. The combination according to claim 30 in the form of a single oral dosage form having about 150 mgs of scyllo-inositol and about 150 mgs of apigenin and pharmaceutically acceptable excipients.
31. A method of supporting cognitive health comprising the administration of a nutraceutically effective amount of a composition comprising scyllo-inositol and apigenin.

### BRIEF DESCRIPTION OF THE DRAWINGS

FIGS. 1A-F shows the effect of 250 mgs scyllo-Inositol Treatment BID in Mild/Moderate AD Patients (MMSE 16-30) in the primary endpoints, NTB, ADCS-ADL, and CDR-SB

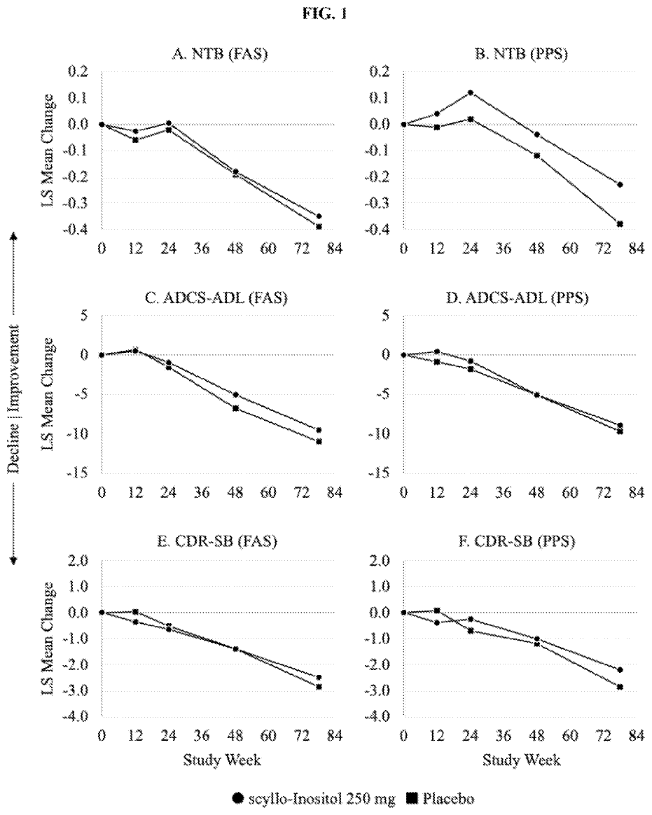

FIG. 2 demonstrates the effect of scyllo-inositol treatment of early mild AD patients (MMSE 23-26) for 78 weeks in a pre-specified full and per protocol populations.

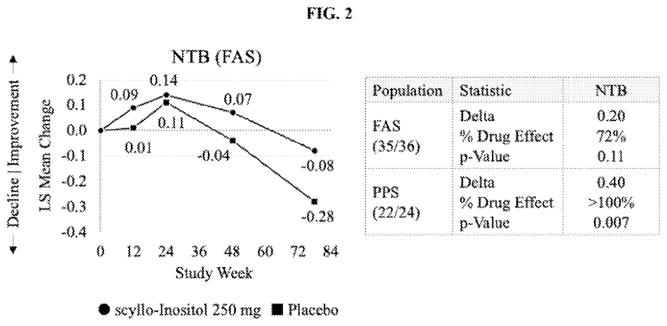

FIGS. 3A-I shows NTB sub-item change from baseline to mild AD (PPS) in nine different sub-items.

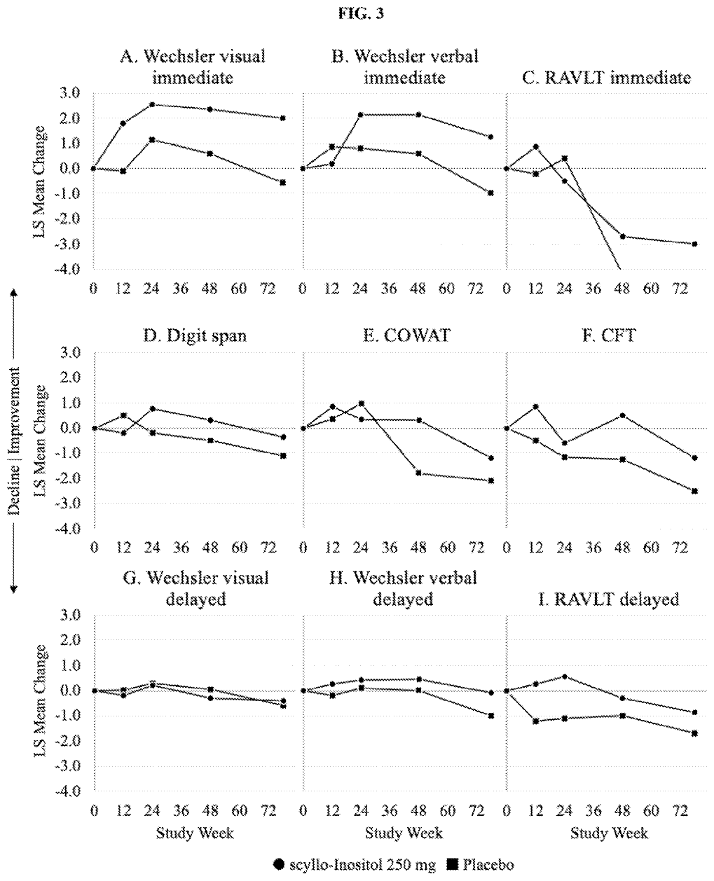

FIG. 4 shows ADCS-ADL change from baseline for scyllo-inositol and placebo treated early-mild AD patients (MMSE 23-26) for 78 weeks.

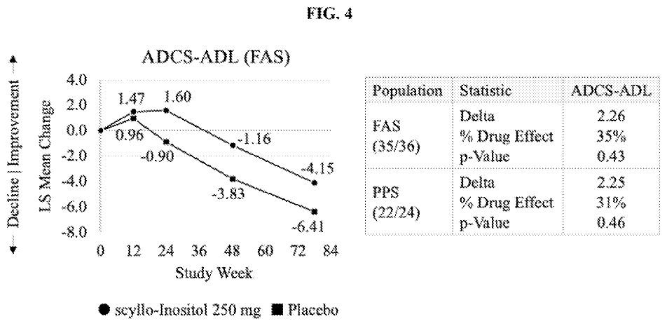

FIG. 5: shows CDR-SB change from baseline for scyllo-inositol and placebo treated early mild AD patients (MMSE 23-26) for 78 weeks.

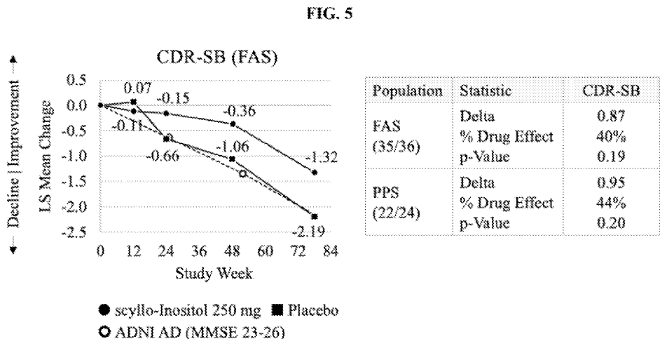

FIGS. 6A-F: shows the compares the effect of scyllo-inositol and placebo treatment on the CDR-SB sub-items change from baseline in early mild AD patients in the per protocol population (PPS).

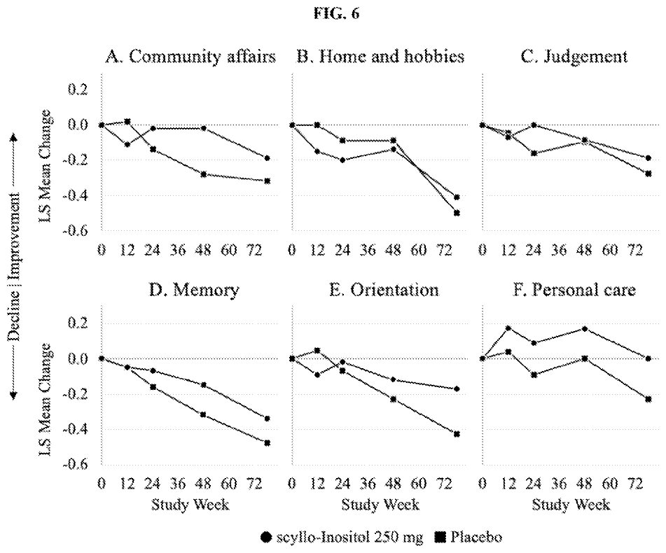

FIGS. 7A-D shows the observed changes from baseline in NTB scores with scyllo-inositol treatment of mild AD patients with different MMSE scores ranging from 20-26, 21-26, 22-26 and 23-26 respectively.

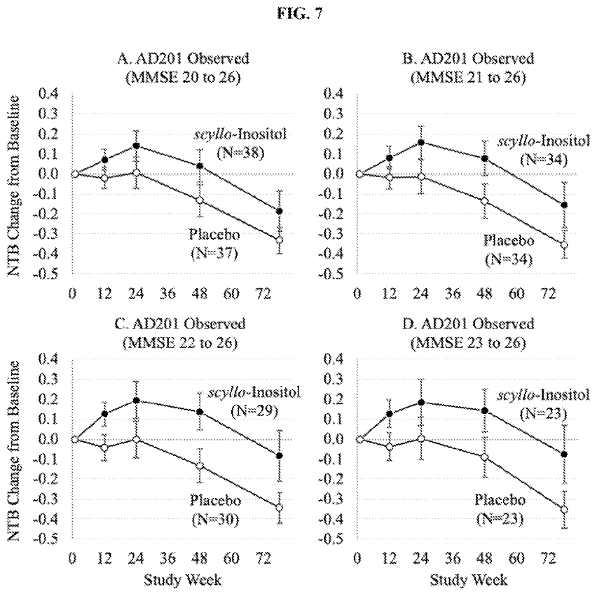

FIGS. 8A-D shows Bootstrap simulated data for the change in NTB scores from baseline with scyllo-inositol treatment of different mild AD patient groups with MMSE scores ranging from 20-26, 21-26, 22-26 and 23-26 respectively.

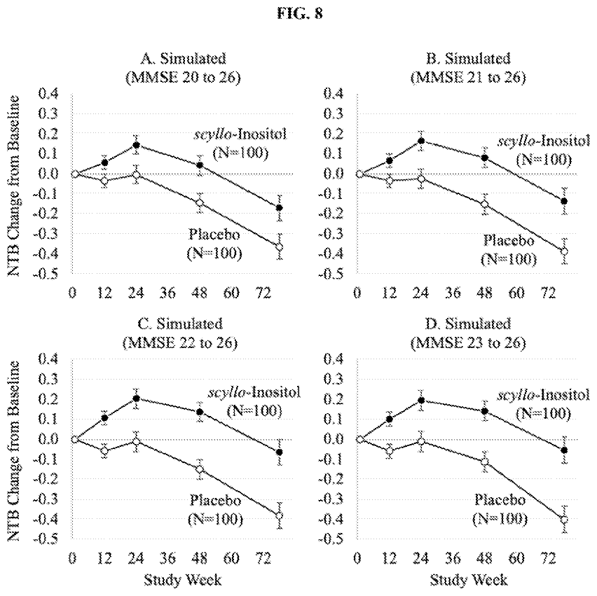

FIGS. 9A-D shows the observed data demonstrating the changes in CDR-SB scores from baseline with scyllo-inositol treatment in different mild AD groups with an MMSE score of 20-26, 21-26, 22-26 and 23-26 respectively.

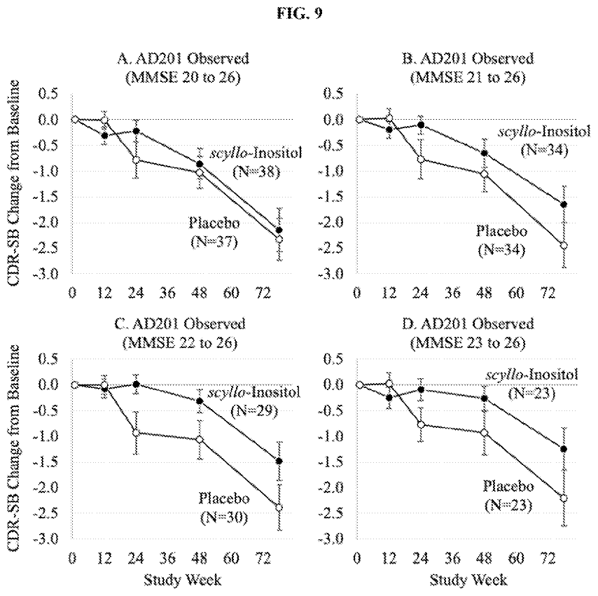

FIGS. 10A-D shows bootstrap simulated data demonstrating the changes in CDR-SB scores from baseline with scyllo-inositol treatment in different mild AD groups with an MMSE score range of 20-26, 21-26, 22-26 and 23-26 respectively.

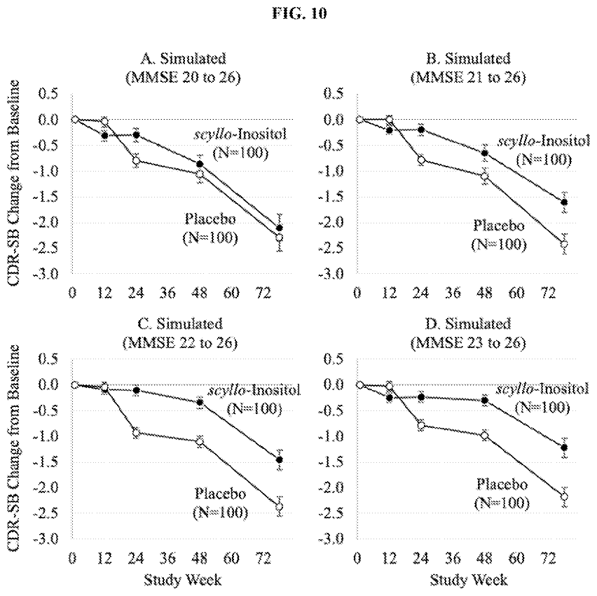

FIGS. 11A-D shows a comparison of Observed and Bootstrap Simulated data for changes in NTB and CDR-SB scores from baseline for mild AD patients treated with scyllo-inositol having MMSE scores of 22-26.

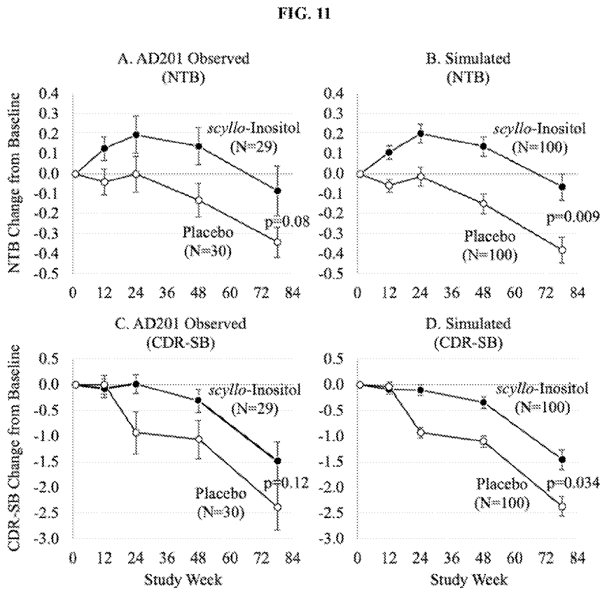

### DETAILED DESCRIPTION

The prevalence of Mild Cognitive Impairment (MCI) and Alzheimer's Disease (AD) increases in the aging population. The gradual loss of memory associated with MCI is followed a further deterioration of memory and increasing loss of function. In late MCI stage, patients begin to develop dementia and convert to mild AD. These patients can progress and develop moderate and severe AD. As shown below accumulation of amyloid beta in the brain occurs early and is pre-symptomatic, as amyloid burden in the bran continues to increase, the onset of early and late MCI appears and continues to progress in severity until the patient is diagnosed with AD. It is well established that onset of clinical symptoms such as memory loss begins in early and late MCI and is followed by a more rapid decrease in memory and function as the patient develops through Mild, Moderate and eventually Severe AD. Patients with late stages of Moderate and Severe Alzheimer's Disease demonstrate reduced function requiring caregiver assistance, and potentially mortality.

Although the exact mechanisms triggering AD pathologies are not well elucidated, it is well established that the accumulation of amyloid beta fibrils resulting in the formation of plaques are the hallmark of Alzheimer's disease. Further, the accumulation of amyloid beta fibrils in the brain begins to occur some 10-15 years prior to the development of dementia and associated clinical symptoms. The accumulation of fibrils and plaques increases with aging where 30-40% of the population over the age of 55, and over 70% of MCI patients have amyloid beta burden in the brain. The presence of amyloid beta fibrils and plaques results in increasing neurotoxicity and appearance of inflammation in the brain. The increase in these cumulative pathologies are associated with the appearance of more aggressive disease and loss of memory and function in AD patients.

Scyllo-inositol is a drug that, in oral form, crosses the blood-brain barrier to achieve low mM levels in the blood. Scyllo-inositol has been reported to break down Aβ fibrils and prevent binding of Aβ to the fibrils. See McLaurin, J. et. al., J Mol. Biol. (1998): 183-194. Scyllo-inositol has been reported in in vitro studies to inhibit Aβ binding to neuronal membrane. (McLaurin J., et. al., J Biol Chem 2000 24:18495) and to restore long-term potentiation in hippocampal slices (Townsend M., et al. Annals of Neurology 2006 December; 60(6):668-76. Scyllo-inositol has been reported to decrease Aβ burden in transgenic AD animal models and to improve cognitive function in such animals. See McLaurin J., et al., Nature Medicine (2006) 12: 801-808. A total of nine phase 1 studies in human trials have been conducted to date. These studies essentially found that scyllo-inositol is orally available; provides a dose proportional increase in plasma levels; crosses the blood brain barrier; achieves levels in the brain and in the CSF fluid that had been shown effective in animal models of AD; and provided an acceptable safety profile that permitted advancing to phase 2 clinical studies. A phase 2 trial of patients on placebo (82 subjects) relative to patients taking 250 mg BID of scyllo-inositol over a 78 week treatment period showed no statistical significance between subjects in the full analysis set in the primary endpoints of NTB and/or ADCS-ADL. Per-protocol sets of patients (placebo=47; scyllo-250 BID=49) showed no statistically significant different in preventing decline at the end of the 78-week treatment period under the primary endpoints measured by NTB and ADCS-ADL. The population of AD patients in the study was mild to moderate Alzheimer's disease having MMSE of 16-26. A publication reported a subgroup analysis of subjects (placebo: FAS 35/PPS 22 versus scyllo 250 Bid FAS 36/PPS 24) in these studies having an MMSE of between 23-26 and even that group did not achieve statistical significance between groups receiving placebo and those receiving scyllo-inositol. See Salloway, et al. Neurology 2011; 77:1253-1262.

While scyllo-inositol's properties with respect to the prevention of Aβ fibril formation along with its positive results on memory/cognition tests in AD animal models have been reported, the published information regarding actual treatment of Alzheimer's disease in subjects have been scarce to non-existent given the fact that the clinical studies to date have been apparent failures, until now. The present inventors have surprisingly and unexpectedly found a subgroup of patients that are amenable to effective treatment with scyllo-inositol to treat the subset of patients having Alzheimer's disease or having MCI while meeting the primary endpoints in the clinical studies. The present invention thus comprises the use of scyllo-inositol alone to treat this subpopulation and/or to provide to healthy subjects in the form of a nutraceutical, which, in a preferred embodiment, comprises a composition comprising a first ingredient selected from scyllo-inositol and a second ingredient selected from an aquaporin 4 upregulator.

### Glossary

Numerical ranges recited herein by endpoints include all numbers and fractions subsumed within that range. (e.g. 1-5 includes 1, 1.5, 2, 2.5, 3, 3.5, 4, 4.5 and 5 and numbers in between such specific numbers).

The term “adjuvant” means a component that, when added to a dosing regimen of a single active ingredient or when added in combination with another active ingredient, such component adds or provides, in combination, an enhanced or beneficial and modified therapeutic or safety benefit to the other active ingredient in the combination when compared to the same properties of the single other active ingredient or component administered alone. The adjuvant on its own may not have clinically significant properties in the target patient population but, in combination with such other active ingredient, does provide additional therapeutic or safety clinically significant properties to such other active ingredient in the target patient population. Adjuvants can be oils known to one of skill in the art.

The terms “administering” and “administration” refer to the process by which a therapeutically effective amount of a compound or composition contemplated herein is delivered to a patient for prevention and/or treatment of the recited condition or disease. The term may also mean administration of a nutraceutical to a subject to supplement the subject with such ingredients.

The term “treating” refers to reversing, alleviating, or inhibiting the progression of a disease, or one or more symptoms of such disease to which such term applies. Depending upon the condition of the patient or subject, the term also refers to preventing a disease, and includes preventing the onset of such a disease depending upon the particular disease or condition.

The terms “subject” or “patient” are used interchangeably herein and include mammalian subjects including humans or animals such as horses, dogs, cows, cats or other mammals.

The term “supporting cognitive health” means the described purpose of the administered dosage form on the label or promotional materials for a nutraceutical product of the claimed invention.

The term “pharmaceutically acceptable excipient or carrier” or “nutraceutically acceptable excipient or carrier” refers to a medium which does not interfere with the effectiveness or activity of an active ingredient or nutritional supplement and which is not toxic to the subjects to which it is administered. Excipients can include diluents, binders, adhesives, lubricants, disintegrates, bulking agents, wetting or emulsifying agents, pH-buffering agents and other known pharmaceutically effective excipients.

The terms “pharmaceutically effective amount” or “nutraceutically effective amount” means the amount of either the pharmaceutical active ingredient(s) or “active agent(s)” present in a nutraceutical which are in the respective dosage form and are distinguished from other ingredients or excipients in the dosage forms that are present in the formulation for purposes other than pharmacological or biological activity.

The term “combination treatment” or “co-administration” means the active ingredients are administered concurrently to a patient being treated. When administered concurrently, each component may be administered at the same time, or sequentially in any order at different points of time. This term is inclusive of pre-treatment with one active ingredient and then treatment with both active ingredients and/or either active ingredient at the same or at different points in time-all in order to achieve the desired therapeutic and/or beneficial effect or supplemental benefit. Beneficial effects include, for example, a diminishment in side effects of one or both active ingredients due to the presence of the other active ingredient.

The term “a beneficial effect” refers to an effect of a compound or adjuvant or composition or combination which includes favorable pharmacological and/or therapeutic effects, and/or improved biological activity and which includes or can include a reduction in side effects. The term “beneficial effects” is inclusive of such effects as improved cognitive function, reduced vascular load, reduced astrogliosis, reduced amyloid burden, reduced microgliosis and/or improved survival. The beneficial effects may also include enhanced stability, a longer half-life, and/or enhanced uptake and transport across the blood brain barrier by one active ingredient or adjuvant to/for the overall benefit of/from the other active ingredient.

Scyllo-inositol, a stereoisomer of myo-inositol, was found to break down and prevent the formation of amyloid beta fibrils in vitro. In vivo, the daily administration of 0.3 to 30 mg/kg of scyllo-inositol to a transgenic mouse model for AD showed a decrease in amyloid beta burden in brain and an improvement of cognition and function testing. Further, treatment with scyllo-inositol showed a reduction in neurotoxicity and brain inflammation. The ability of scyllo-inositol to cross the blood brain barrier through the myo-inositol transporters allows the drug to achieve sufficient levels to reduce large amyloid aggregates and plaques to small oligomers of amyloid beta. The improvement in cognition may reflect the decrease in amyloid burden and reduction on large aggregates and plaques.

Preclinical studies were conducted as disclosed in, for example, US2007/0197452, using methods that tested Alzheimer's disease mouse models such as TgCRND8 mice. The tests conducted include behavioral tests such as Morris Water maze testing; cerebral amyloid burden; plasma and cerebral Aβ content; gliosis quantitation; survival census; analysis of APP in brain; soluble Aβ oligomer analysis; long term potentiation; and synaptophysin immunohistochemical staining. The results obtained in such studies demonstrated the effectiveness of scyllo-inositol in treating TgCRND8 mice having morphology, density and distribution of amyloid plaques similar to those seen in the brains of human patients having Alzheimer's disease.

### Scyllo-Inositol

Scyllo-inositol may be obtained from processes disclosed in a number of patents and applications. See U.S. Pat. Nos. 8,409,833 and/or 7,745,671, both incorporated by reference herein. Its use in preventing, treating and diagnosing disorders of protein aggregation is disclosed in, for example, EP1608350B1 or U.S. Pat. No. 8,859,628 or 7,521,481, which are incorporated by, reference herein. The data shown therein demonstrated that scyllo-inositol treatment in mice significantly decreased amyloid burden and gliosis. Scyllo-inositol is described as having the property of inhibiting already established amyloid deposition in the living brain. Thus, the data suggests that scyllo-inositol has the properties, in mammals in need of treatment thereof, of reducing amyloid plaque burden and improving cognition. Diseases that can be treated with scyllo-inositol include conditions of the central or peripheral nervous system or systemic organs and which have, as a condition, deposits of proteins or protein fragments and peptides in beta-pleated sheets and/or fibrils or aggregates. Scyllo-inositol provides advantages that include (1) the ability to cross the blood brain barrier to break down amyloid fibrils, reduce amyloid burden in the brain; (2) reduce the aggregation of Ap.

There is basis to believe that very early intervention of pharmaceutically effective or nutraceutically effective amounts of scyllo-inositol, alone or in combination with aquaporin-4 upregulators, can be provided to subjects to reduce fibril formation. Scyllo-inositol also prevents AP aggregation, reduces Aβ burden and improves cognitive function in transgenic animal models for Alzheimer's disease. See McLaurin J., et al. J Biol Chem 2000 24:18495; McLaurin J., et al., Nature Medicine 2006 July; 1247): 801-8; and Townsend M., et al., Annals of Neurology 2006 December; 60(6):668-76.

Scyllo-inositol may be formulated into any suitable pharmaceutical formulation. The compound may be delivered orally or by other suitable means. Oral formulations may be in the form of tablets or capsules, which include pharmaceutically acceptable excipients selected from binders, fillers, surfactants, preservatives, lubricants and the like. The amount of the drug varies but, in the combination treatment, it is between 75 to 250 mgs BID or 500 mg QD. This dosage amount can vary depending upon the particular combination product and subjects being provided with, for example, a nutraceutical composition or product. The tablets and/or capsules may be manufactured by means known to those of skill in the art. The administration of scyllo-inositol may also be accomplished by the use of oral liquids or suspensions, intravenous administration, intramuscular administration or other means such as intraperitoneally, intradermally, transcutaneously, subcutaneously, intranasally, sublingually, inhalation or other means. Adjuvants may be added to enhance delivery of the bioactive agent. In an embodiment, the invention comprises a composition comprising scyllo-inositol, apigenin and an adjuvant.

The preferred combination products are oral dosage forms. Oral solid dosage forms contain active substances such as scyllo-inositol and apigenin with suitable diluents. The dosage forms can be prepared by compression or molding methods. The tablets can be prepared from powdered, granular or crystalline materials alone or in combination with pharmaceutically or nutraceutically acceptable excipients such as binders, disintegrants, lubricants, diluents and colorants or flavors. Diluents may be selected from, for example, dicalcium phosphate, calcium sulfate, lactose, cellulose, kaolin, mannitol, sodium chloride, dry starch and powdered sugar. In some cases and in some formulations, tablets may be formed that disintegrate and, in other cases, tablets may be formed that do not rapidly disintegrate. Microcrystalline cellulose may be used in direct compression formulations. Binders may be selected from the group consisting of starch, gelatin or sugars selected from sucrose, glucose, dextrose, molasses and lactose. Natural and synthetic gums selected from sodium algainate, acacia, panwar gum, methylcellulose, polyvinylpyrrolidone may be used as binders. Lubricants include talc, magnesium stearate and the like. Glidants may be selected from silicon dioxide and the like. Disintegrants may be selected from starches, clays, celluloses, aligns, gums and cross-linked polymers.

Powder compaction or wet granulation methods may be used to form tablets. The active ingredients, diluents and disintegrants are mixed then dried, screened, lubricated and pressed. Dry granulation methods may also be used to form tablets. Additional methods include spray drying and spheronization.

Other suitable oral dosage forms include capsules. Capsules can include hard and soft gelatin capsules or soft capsules containing gelatin substitutes. Capsule filling methods are known in the art. Hard gelatin capsules are easy to fill and could be particularly useful for a combination of scyllo-inositol and apigenin. The two active ingredients can be blended together with pharmaceutically or nutraceutically acceptable excipients to form fine, uniform powders. The two active ingredients may also be formed into two separate capsules having, for examples, two different colors for packaging in a kit or capsule container containing both dosage forms. Having the two ingredients in a single dosage form has numerous advantages including ease of use and formulation advantages.

Coatings may also be applied to the tablets or capsules produced having either or both of scyllo-inositol and apigenin. The coatings may serve taste masking purposes or be utilized to make functional embodiments such as delayed release capsules or tablets.

For the compositions and methods described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description, taken in conjunction with the drawings. While the compositions and methods are susceptible of embodiments in various forms, the description hereafter includes specific embodiments with the understanding that the disclosure is illustrative, and is not intended to limit the invention to the specific embodiments described herein.

In one aspect, the combination formulations comprise scyllo-inositol and a flavone component. In another aspect, such formulations are in the form of spheronized pellets or multiparticulates. In one aspect, the formulations comprise spheronized pellets comprising an excipient selected from the group consisting of binders, surfactants, diluents, lubricants, gums, waxes, polymers and the like. The polymers may be selected from a water-insoluble polymer, and can optionally include a water-soluble polymer. In an aspect, the formulation can be a nano/micro particle formulations, e.g. made by emulsion and spray drying, such as an emulsion-diffusion-spray drying freeze drying technique described herein. In another aspect, the formulations can be a powder formulation, e.g. made by hot-melt cooling/spray congealing. In another aspect, the formulation can be coated seed. In another aspect, the formulation can be an active-containing granule.

Spheronized pellets produced from aqueous-based wet granulation processes can also have other components in the granulation blend including absorption enhancers, diluents and spheronizing aids, pore formers, binders, binding aids, fillers and water.

The formulations and dosage forms, and their related methods of making and are contemplated to include embodiments including any combination of one or more of the additional optional elements, features, and steps further described below, unless stated otherwise. Oral suspension formulations may also be made containing scyllo-inositol and apigenin or other agent that increases the expression of aquaporin 4 or otherwise increases either channel number or clearance through such channels.

As used herein, the term wt. % refers to parts by weight based on the total weight of the thing described, for example based on the total weight of the vitamin-D containing region, by default, or based on the total weight of the formulation, where applicable by context or explicitly described.

One aspect of the disclosure herein is a formulation, comprising an active pharmaceutical ingredient selected from scyllo-inositol and apigenin dispersed in a rapidly releasing pharmaceutical composition and which is provided in hard capsule form. In embodiments, the formulation is an immediate release formulation for oral use.

Another aspect of the disclosure herein is a nano/microparticle formulation comprising scyllo-inositol and apigenin and, optionally an additional active pharmaceutical ingredient, and a combination of pharmaceutically acceptable excipients or nutraceutically acceptable excipients. In embodiments, the nano/microparticle formulation can provide immediate release of the combination of scyllo-inositol and apigenin in dosage amounts of about 70-125 mgs or ratios by weight of about 1:1 or 1:1.5 or 1:1.6 or 1:1.7 or 1:1.8 or 1:1.9 or 1:2, e.g. by using a blend of excipients.

Another aspect of the disclosure herein is a non-pareil seed formulation comprising an active pharmaceutical ingredient, preferably scyllo-inositol alone or in combination with apigenin and a pharmaceutically acceptable excipient. In embodiments, the formulation can be an immediate release formulation, e.g. for oral use. In embodiments, any one of the hard capsules can include an additional polymer coating. Such coatings may be on the pellets or granules or seeds within the capsule and/or the capsule may be externally coated.

In one aspect, extrusion-spheronization is utilized to produce pellets of good physical strength, uniform diameter and good porosity. The main advantage of pellets prepared by extrusion-spheronization method over other methods is the ability to incorporate high drug loads, in particular high drug loads of scyllo-inositol and, optionally apigenin, without producing overly large particles.

It is thus possible to manufacture multiparticulates by extrusion-spheronization having an active ingredient in conjunction with a number of other excipients assisting the wettability of the formulation, the release profile and the ability to create extrudates and spheroids, which are suitable for oral delivery of such ingredients. The drug loading achieved for suitable strengths of drug in these pellets or multiparticulates comprises at least about 50-90% of the total weight of the formulation excluding the capsule. An optional coating system may also be provided on the surface of the multiparticulates. This allows for safe handling of the active baring multiparticulates and for easy filling into capsules, stick-packs or suitable containers for bulk supply. The preferred active combination is scyllo-inositol and apigenin.

Another aspect of the disclosure herein are methods of making the pharmaceutical formulations and dosage forms. In embodiments, the method can be a method of making an immediate release pharmaceutical formulation, comprising compounding an active pharmaceutical ingredient such as scyllo-inositol and, optionally, apigenin with at least one excipient selected from the group consisting of spheronizing aids (diluents), humectants, stabilizers, binders, superdisintegrants, lubricants, solvents and optional coatings.

The invention further comprises a process which comprises blending an API, a diluent/spheronizing aid and a superdisintegrant and dispensing such materials and, adding binders, optional humectants and granulating/wet massing under certain conditions and endpoints; extruding the wet mass; spheronizing the screened extrudate; drying; fractionizing; adding lubricant and optionally coating to form a formulation having a uniform size and shape suitable for filling capsules, sachets and/or stick packs. In a preferred embodiment, the API is selected from a combination of scyllo-inositol and apigenin.

Another aspect of the disclosure herein is a method of treating a disease or condition comprising administering a formulation or dosage form according to the disclosure herein to a subject in need thereof. In a preferred embodiment, the subject is a mammal, including humans and animals such as dogs.

Another aspect of the invention herein is a method of supporting cognitive health in a subject in need of support thereof comprising administering an amount of the combination of scyllo-inositol and apigenin in a suitable dosage form to said subject. The dose, strength and relative ratio of the actives can be varied depending upon the cognitive disorder or condition in the subject. In some cases and in some situations, the preferred combination is a nutraceutical product that is provided to subjects years before any sign or evidence of cognitive impairment such as memory loss is present in the subject. It is believed that the provision of such products or supplements will help to prevent the subsequent aggregation of beta amyloid and such products will facilitate clearance of fibrils through the glymphatic system. Pharmaceutical and clinically effective products are also within the scope of the claims presented in the disclosure.

Methods utilized to measure clinical efficacy and outcomes are determined on a patient-by-patient basis and which involve measuring and determining the existence, severity and progression of Alzheimer's disease over a period of time. This involves clinically determining the global functioning level of the patient; daily living behavior and deficits in living capacity or behavior; volumetric analysis of brain structures and in vivo measurements of disease related deposits of abnormal proteins in the brain using techniques such as PET imaging for beta amyloid proteins. In addition, the measurement of blood, body fluid or CSF markers as an indicator of the presence of disease or progression of disease is also performed and includes measurement of tau proteins and other biomarkers such as pyroglutamate-Aβ, Aβ40 and Aβ42 in blood as well as total Tau, phosphor Tau, pyroglutamate-Aβ, Aβ40 and Aβ42 in CSF. ApoE isotype as well as hippocampal volumetric (ICV) MRI are useful as well to define and/or stage disease progression. The measurement of such markers and methods to determine such marker levels are known in the art. In addition, it is known that such markers can be predictive of pathogenesis in Alzheimer's disease. See, for example, Duyckaerts (2011) Lancet Neurol. 10, 774-775. And Craak, et al., (2013), Acta Neuropath., 126:631-41.

Amyloid plaque burden is measured by 18F-AV45 PET. 18F-AV-45 is a known amyloid ligand sold and developed by Avid Radiopharmaceuticals. The skilled PET imaging specialist can review the PET images obtained to determine the mean uptake of 18F-AV-45 in and between AD patients and age-matched control subjects. PET measurements of regional glucose metabolism and morphometric MRI measurements are also utilized to assess AD state or progression. MRI is monitor ARIA related events.

U.S. Pub. No. 2021/0401752, hereby incorporated by reference, discloses dosage forms of calcifediol dispersed in a polymer composition, which can be in an extended release formulation. The embodiments include a vitamin D compound such as calcifediol embedded in a polymer network. The specification discloses spheronized pellet formulations comprising 25-hydroxyvitamin D and a pharmaceutically acceptable excipient. In certain embodiments, the spheronized pellets comprise an extended-release component selected from a polymer and/or lipid component. The polymer may be a water-insoluble polymer and can include or be a water-soluble polymer. The formulations can be a nano/micro particle formulation made by emulsion followed by spray drying freeze drying techniques as described in the '752 publication. The formulations can also be powder formulations made by spray congealing. The formulations can include an extended release coated seed or an active coated granule. The formulations can be free of waxes and/or include waxes.

Immediate release dosage forms may be prepared according to, for example, AU 2021100513, which discloses an immediate release tablet formulation of the active, disclosed therein. There a spray-dried powder prepared from an emulsion is added to tablet excipients and formed into a tablet having 10 micrograms of the active ingredient. This process can be applied to produce a dosage form comprising scyllo-inositol in combination with apigenin.

The combination of the flavone apigenin (C₁₅H₁₀O₅) in dosage amounts ranging from about 10 mgs to about 500 mgs and scyllo-inositol (100-500 mgs) may be utilized in separate dosage forms or in a single dosage form having both ingredients. In a preferred embodiment, the dosage form is a single dosage form such as a tablet or capsule having about 150 mgs of each active. The combination along with other pharmaceutically acceptable excipients comprises a pharmaceutical or nutraceutical composition useful for the treatment of cognitive disorders, mild cognitive impairment and the treatment of Alzheimer's disease in those subset of patients having MMSE scores of about 22 to 26. Apigenin is commercially available and is sold as a nutritional supplement in a range of doses.

The combination of scyllo-inositol and apigenin will reduce the formation of amyloid beta aggregates and will increase amyloid beta clearance more than either drug administered alone at the same dose or strength. The combination provides improvements in people having memory and cognition loss associated with aging subjects with early to mild AD and the combination of reducing aggregation and increasing or facilitating clearance can help minimize or reduce brain amyloid accumulation and improve memory loss and cognitive impairment in the treated population. Scyllo-inositol blocks amyloid fibril formation and breaks down amyloid beta oligomers and fibrils and increases clearance and the reduction of amyloid burden in the brain. Apigenin increases the expression and localization of aquaporin-4 channels on perivascular astrocytes responsible for clearance of waste, including clearance of amyloid beta. The combination reduces amyloid beta accumulation in the brain and this reduces the risk of progression of cognitive impairment and dementia, and improves memory and cognition in the aging population.

Aquaporin-4 channels are involved in the clearance of amyloid beta n the brain and deletion of these channels results in increased memory deficits in mouse AD models. Xu, et al. Mol Neurodeneneration 2015; Sapkora, et al. Brain 2022. Apigenin reduces the half-life of amyloid beta in brain interstitial fluid, increases amyloid beta clearance, and improves cognition in a mouse AD model. Zhao, et al. 2013.

The prevalence of amyloid beta accumulation increases with aging and may occur 10 to 15 years prior to the onset of memory loss, mild cognitive impairment and Alzheimer's disease. Thirty to forty percent of subjects over the age of 55 have amyloid beta accumulation in the brain, and over 70% with mild cognitive impairment. The increase in accumulation of amyloid beta in the aging brain is most likely due to the aggregation and formation of amyloid beta oligomers and fibrils, which become trapped and not easily released into the CSF or digested by cells or enzymatically in the brain. Once amyloid beta precursor protein is synthesized and appears on the neuronal cell surface, it is cleaved by alpha and beta secretases, producing free amyloid beta peptide. The free amyloid beta peptide is cleared through glymphatic system on the perivascular astrocytes into the CSF. In addition, microglia can take up and digest free amyloid beta. The combined clearance mechanisms ensure a low concentration of amyloid beta in the brain interstitial fluid and prevents the formation of amyloid beta aggregates and fibrils. Any increase in amyloid beta synthesis combined with an imbalance with either of these clearance mechanisms can result in the accumulation of amyloid beta and an increase in the loss of memory and cognition.

Ageing, cerebrovascular disease, prior exposure to repeated concussions or other traumatic brain injuries, sleep disruption and genetic predisposition are emerging risk factors for the development of neurodegenerative conditions, such as Mild Cognitive Impairment and Alzheimer's Disease. In animal models and human Alzheimer's Disease, the glymphatic function in brain responsible for clearance of waste, including amyloid beta may be impaired. It has been demonstrated that subjects with Alzheimer's Disease have reduced aquaporin channels at the perivascular astrocytes involved in glymphatic exchange in amyloid-O and tau clearance. The reduced clearance of amyloid beta may play a key role in the accumulation of amyloid burden and related development of these conditions. In post-mortem case series, reduced perivascular AQP-4 channels was observed in the frontal cortex of AD subjects, while they were preserved in matching aging patients with normal cognitive impairments over the age of 85. Thus, it is possible the reduced aquaporin 4 channels result in an impairment in the clearance of amyloid beta, tau and other waste in the brain through the CSF. These findings suggest that changes in expression and localization of aquaporin channels may contribute to the development and progression of neurodegenerative diseases. Thus, it is likely that a combination of disaggregation of amyloid fibrils in brain and increased expression and localization of aquaporin channels in perivascular astrocytes will be beneficial in the clearance of amyloid beta and tau through the CSF.

In a post-mortem case series, reduced perivascular AQP4 abundance was observed in the frontal cortex of subjects diagnosed with Alzheimer's disease, while preservation of perivascular AQP4 abundance was observed in subjects remaining cognitively intact over the age of 85. The reduced perivascular AQP4 abundance was further associated with increasing amyloid-β and tau pathology, as well as with global measures of cognitive decline. In three recent genetics studies carried out in distinct human populations, single nucleotide polymorphisms in the human AQP4 gene were associated with variation in cognitive decline, amyloid burden and clinical status, and an association between sleep disruption and amyloid burden. A recent human transcriptomic study further demonstrated that in addition to the expression of AQP4, differences in the expression of genes whose products determine perivascular AQP4 localization (specifically genes encoding elements of the DAC, SNTA1, DTNA, DM), DAG1) were associated with dementia status and temporal cortical tau pathology. These findings suggest that changes in AQP4 expression and localization may contribute to the development and progression of neurodegenerative diseases, including Alzheimer's disease, in human populations. Understanding the emerging role of dynamic AQP4 subcellular relocalization provides a new framework to understand waste clearance in the healthy brain and opens up new treatment avenues to slow the progression of neurodegenerative diseases.

The combination of scyllo-inositol and apeginin is designed for the aging population at risk for amyloid beta accumulation in brain that may potentially result in memory loss and cognitive impairment. Scyllo-inositol prevents the formation of amyloid beta aggregates in the brain and breaks down existing aggregates to produce amyloid beta monomers, which can be taken up and digested by microglia as well as cleared by the aquaporin channels on perivascular astrocytes into the CSF. In comparison, Apigenin increases the clearance of amyloid beta in brain by increasing the expression and localization of aquaporin 4 channels on perivascular astrocytes responsible for clearance of amyloid beta, tau and other waste through the CSF. By combining both natural products. The combination product will enhance the clearance of amyloid beta in the brain and help prevent the onset and progression of memory loss and cognitive impairment.

The benefits of the combination of scyllo-inositol and apigenin include:
reduced amyloid beta accumulation and plaque formation in brain
improvement of memory and cognition
reduced risk of progression of cognitive impairment and dementia with aging
both scyllo-inositol and apigenin have a good safety profile.

Scyllo-inositol is a natural sugar found in many nutrients, including coconut, carrots, grapes and citrus fruits. It is well established that scyllo-inositol prevents the formation of amyloid beta aggregates and fibrils in vitro in a concentration dependent manner. 0.1 uM to 5 uM of scyllo-inositol prevents the binding of amyloid beta monomers to amyloid beta oligomers, and prevents the formation of oligomers and fibrils. In addition, similar concentrations of scyllo-inositol breaks down existing amyloid beta fibrils into soluble monomers.

Amyloid beta binding to neurons results in reduced synaptic activity and neurotoxicity. Scyllo-inositol has been shown to prevent amyloid beta binding to neurons and reduced neurotoxicity (Maclaruin, et al.). Further, Townsend, et al. have shown that scyllo-inositol infusion in rat brain restores long term potentiation induced by toxic amyloid beta oligomers in hippocampal slices and restored memory loss induced by amyloid beta in a rat model.

Mclauhrin, et al. examined the efficacy of scyllo-inositol in vivo in a Alzheimer transgenic mouse model. Animals were treated orally with doses of 0, 3, 10 or 30 mg per kilogram of scyllo-inositol for a period 4 months following the onset of amyloid beta fibril and plaques. The brains of each group were examined for amyloid beta fibrils and plaques, amyloid beta burden and cognition. Treatment with scyllo-inositol with doses as low as 3 mg/kg were shown to reduce the staining for amyloid beta fibrils and plaques, as well as amyloid beta burden. Evaluation of cognition using a Morris Water maze test showed that untreated Transgenic AD animals showed a deterioration in cognition with the accumulation of amyloid beta in brain. However, treatment orally with scyllo-inositol reduced amyloid beta burden sufficiently to improve cognition that was comparable to normal animals.

In summary, the AD model study showed that orally administered scyllo-inositol inhibited Alzheimer's Disease-like behavioral deficits, neuropathology and accelerated mortality in the AD transgenic mouse model of Alzheimer's disease. These effects were observed regardless of whether the scyllo-inositol was administered during the onset of disease or during the overt phase of AD-like disease.

A phase 2 study examining the effect of scyllo-inositol on Mild and Moderate AD patients.

A randomized, double blind placebo-controlled study phase 2 study explored the safety and efficacy of 250 mg BID administered to mild and moderate AD patients over 78 weeks. One of the primary endpoints used to measure changes in cognition was the Neuropsychological Test Battery (NTB) as well as measuring the levels of biomarkers in the CSF such as Amyloid beta 42 and phosphorylated Tau. The primary analysis compared 250 mg BID (=−84) and placebo (n=82) in mild to moderate patients, and an additional pre-specified analysis was performed in the placebo and treated mild AD patients. Changes in cognition were not statistically significant in the mild to moderate patient population. However, there was a very strong signal in improvement of NTB score in the mild AD patients treated with 250 mg BID (See Figure xxx below). Interestingly, scyllo-inositol showed improvement in 7 of the 9 parameters measured in the NTB. Similar trends were also observed in other cognition and function tests such CSR-SoB and ADCS-ADL. The effects of scyllo-inositol were more pronounced in the per protocol population that received at least 80% of the medication over the 78 weeks.

The glymphatic system plays a key role in the clearance of waste in the brain, including amyloid beta and tau. More specifically, the aquaporin-4 channels are critical in the ongoing clearance of amyloid beta from brain interstitial fluid to the CSF via the perivascular astrocytes. The most convincing evidence comes from the transgenic mouse models with the aquaporin 4 gene deletion. When the Aquaporin 4 gene is deleted in transgenic AD mouse model, the % of plaques increase significantly in both the cortex and hippocampus compared to the transgenic mouse model. Further, deletion of the aquaporin 4 channel gene in both wild type and transgenic AD mice, showed a deleterious impact on cognition. These data clearly show that Aquaporin channels are essential to clear amyloid beta and retain normal cognition in normal and AD brain in mouse models. The impact of reduced aquaporin 4 channel levels have a more pronounce effect on cognition in AD brain.

More recently, it has been shown that AD human brain has a decrease in localization of Aquaporin 4 channels localized in on the perivascular astrocytes responsible for clearance of amyloid beta. It may not be the expression of aquaporin 4 channels but rather an impairment in the localization of the aquaporin channels on the perivascular astrocytes that may be responsible for the decrease in amyloid beta clearance resulting in accumulation in the brain. Patients with decreased localization of aquaporin channels on perivascular astrocytes correlated with increased amyloid burden relative to aging subjects with normal cognition. These data suggest that agents, which can increase expression and localization of aquaporin channels in perivascular astrocytes, may be beneficial for the reduction of amyloid burden in the brain of aging patients at risk with amyloid beta accumulation.

Zhao, et al. demonstrated that apigenin treatment decreased the accumulation of amyloid beta in transgenic AD mice and reversed the loss in cognition to normal levels. The question is what is the mechanism of action for apigenin responsible for decreasing amyloid beta accumulation.

More recently, Sapkota, et al. demonstrated that apigenin treatment in Transgenic AD mice significantly reduced the amyloid beta half life, suggesting an increase in amyloid beta clearance in AD brain. These effects were not observed in Transgenic AD mice with deleted Aquaporin 4 gene suggesting that the effect on clearance with apeginin may be through the aquaporin channels. Further, this publication also demonstrated that apigenin treatment in transgenic AD mice increased the expression and localization of aquaporin channels on perivascular astrocytes. These findings are suggestive that Apeginin can increase clearance of amyloid beta and Tau through the glymphatic system to reduce amyloid beta accumulation occurring in brain of aging population at risk of cognitive impairment.

The administration of the combined product comprising scyllo-inositol and apigenin will act to disaggregate oligomers of amyloid beta thus increasing clearance in the brain. A composition comprising scyllo-inositol and apigenin at suitable doses selected from a dose ranging study in mice has the potential to prevent cognitive impairment and neurodegenerative diseases related to the amyloid beta burden in the aging population 5 to 10 years prior to a deficit in cognitive symptoms.

Both Scyllo-inositol and apigenin have been administered in animals and humans previously. The safety profile is acceptable for long-term use.

### Scyllo Inositol: Preclinical

The full preclinical safety program for scyllo-inositol has been completed. The data shows the scyllo inositol administered in rats up to 2500 mg/kg/day for 6 months was safe with no observed pathologies. In addition, dogs treated with 300 mg/kg/day for 12 months was safe and no pathologies. At 2000 mg/kg/day for 12 months, some mild adverse events were observed such as skin inflammation, gingivitis and pneumonia.

### Scyllo-Inositol: Clinical Studies

The clinical development program of ELND005 has to date included a total of 18 studies, of which 6 are currently ongoing and 1 recently initiated.

### Completed Studies

A total of 13 clinical studies with ELND005 have been completed and include nine Phase 1 studies, two Phase 2 studies in patients with Mild to Moderate AD dementia, one phase 2 study on agitation and aggression in moderate to severe AD patients and one phase 1/2 study in Down's Syndrome.

In the completed studies, over 470 subjects have been exposed to at least a single dose of ELND005 (161 unique healthy subjects in Phase 1 studies, 306 unique patients in Phase 2 studies). The 306 Mild and Moderate AD patients were treated with ELND005 doses of up to 2000 mg BID. In the completed Phase 2 studies, the duration of treatment was up to 2.5 years with the 250-mg BID dose. The Phase 2 AD studies provided a total of 370.2 patient-years of exposure at any dose. At the dose of 250 mg BID, there have been a total of 154.8 patient-years of exposure in AD patients. In phase 2 studies in agitation and aggression in moderate to severe AD patients, over 150 patients were treated with 1000 mg BID for 4 weeks and then maintained with 250 mg scyllo-inositol BID for 12 weeks.

Of the 161 healthy subjects who received ELND005 in Phase 1 studies, 87 were between the ages of 18 and 54 years (inclusive) and 74 were between ages of 55 and 81 years (inclusive). The dose range was 70 to 7000 mg in single-dose studies and 200 to 3000 mg BID in multiple-dose studies for up to 10 days. Patients with AD were treated with 250 mg BID to 2000 mg BID.

Based on the review of clinical studies of up to 2.5 years by the clinical monitoring committee and agreement with the FDA and EMA for to perform additional clinical trials, 250 mg BID of scyllo-inositol is considered safe for long-term use.

### Apigenin

Apigenin (4′,5,7-trihydroxyflavone or 7-trihydroxyflavone) is available in many different foods such as parsley, chamomile, celery, vine-spinach, artichokes and oregano. The richest sources are in dry forms, for example dry parsley has been reported to have a maximum of 45 mg/gm of dry parsley. Thus, subjects are exposed to high levels of apeginin in their daily diet. Apigenin is currently available as a dietary supplement in 50 to 300 mg capsules to be taken daily. Apigenin is described as a flavonoid with low toxicity and has numerous bioactive properties. In addition to its effect on aquaporin 4 receptors, it can prevent cell mobility, stimulate the immune system and maintain the cell cycle. It has been described as possessing anti-inflammatory properties. See Abid et al. Molecules 2022, 27, 4304. Apigenin has reduced water solubility and is lipophilic. The chemical structure is:

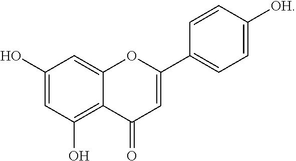 [SMILES: O=c1cc(-c2ccc(O)cc2)oc2cc(O)cc(O)c12]

Apigenin has been described as having the ability to protect neurites and cell viability by promoting the downregulation of cytokine and nitric oxide (NO) release in inflammatory cells. In addition to having beneficial properties on aquaporin 4 expression, it also acts through different mechanisms to provide neuroprotective effects in mammals and in human induced pluripotent stem cell (iPSC) models of familial and sporadic Alzheimer's disease. See Balez, et al. Scientific Reports 6:31450 or www.nature.com/scientificreports, Aug. 12, 2016. Levels of apigenin as low as 3.5-4.5 mgs per day for periods of days to weeks can result in elevated levels of glutathione reductase and superoxide desmutases. Persons who consume vegetables or plants having significant quantities of flavones such as apigenin can also take a combination product of scyllo-inositol that has lower doses of apigenin while persons who consume fewer plant based foods may select a combination of scyllo-inositol having a higher dose of apigenin in the combination product. Thus, in one embodiment, the present invention comprises a tablet or capsule having about 75-150 mgs of scyllo-inositol and about 4-50 mgs of apigenin. Table 1 provides a range of combination products suitable for a consumer that can select the ratio based upon their particular diet.

TABLE 1

| Scyllo-inositol | apigenin |
|---|---|
| 75 mgs | 4 mgs |
| 75 mgs | 10 mgs |
| 75 mgs | 15 mgs |
| 75 mgs | 20 mgs |
| 75 mgs | 25 mgs |

In another embodiment, a dosage form having both scyllo-inositol and apigenin may be selected from a dosage form having a higher strength of scyllo-inositol such as 150 mgs with the same 4-25 mg variation in 5 mg increments or another higher strength apigenin line having increments of 10, 15, 25, 50 and 75 mgs of apigenin. Similarly, the dosage forms having 75 mgs of scyllo-inositol may have the higher strength range of apigenin depending upon the subjects dietary intake.

Other flavones or flavonoids may also be combined with scyllo-inositol to provide beneficial effects. The additional flavones or flavonoids are inclusive of the plant polyphenols selected from the group consisting of kaempferol, quercetin, myricetin and luteolin. These plant polyphenols are widely available. These additional polyphenols may also be combined with a composition comprising scyllo-inositol and apigenin.

Preclinical studies in animals have used 20-40 mg/kg/day over 3 to 4 months of treatment. No adverse events were observed or described.

Clinical studies using either apigenin in a capsules or different food sources have been evaluated for safety. No signs of adverse events observed.

In vitro studies demonstrated that 0.2 to 5 uM or approximately 2-5 ugms/ml of scyllo-inositol is sufficient to prevent formation of oligomers and break down amyloid beta aggregates and fibrils. Thus, our target concentration in the CSF (which we assume to be similar to the interstitial fluid in the brain) is approximately 2-5 ugm/ml.

Administration of 250 mg BID of scyllo-inositol to AD patients results in average scyllo-inositol levels in CSF of 15 ug/ml. If our target does is approximately 2-5 ug/m of scyllo-inositol, then the dose would be approximately 75 mgmg of scyllo-inositol daily up to the 250 mg BID previously shown to improve cognition in mild AD patients.

Further the effective doses of scyllo-inositol in transgenic AD mice was approximately 3 to 30 mg/kg/day dose. If translated into humans at equivalence taking into consideration in the sensitivity between humans and mice (9:1), then it would be approximately 20 to 200 mg of scyllo-inositol daily for a 60 kg subject.

Thus, scyllo-inositol doses of 75 to 250 mg daily should be sufficient for efficacy and safe as described above.

The efficacy of apigenin observed in animal models is approximately 20-40 mg/kg/day. If one assumes that mice are approximately 9 times more sensitive based on weight, then approximately 2-4 mg/kg of apigenin would seem appropriate for efficacy. Thus, an appropriate dose of apigenin in an adult human subject would be approximately 120 to −240 mg/day.

In an embodiment, the claimed invention comprises a pharmaceutical composition comprising a first active ingredient selected from scyllo-inositol and a second active ingredient selected from apigenin.

Clinical assessments employed to determine the stages of Alzheimer's disease and overall progression and/or improvement in stopping or ameliorating disease progression use CDR, FCSRT, Neuropsychiatric Inventor-Questionnaire (NPI-Q), and neurological test batteries which include Rey Auditory Verbal Learning Test (RA VLT), Immediate and Delayed Recall, Wechsler Memory Scale (WMS) Verbal Pair Associate Learning Test Immediate and Delayed Recall, Delis-Kaplan Executive Function System Verbal Fluency Conditions 1 and 2, and the Wechsler Adult Intelligence Scale Fourth Edition Symbol Search and Coding Subsets and Cognitive Drug Research test battery. Mini Mental Test Scores MMSE and Neuropsychiatric Batter test (NTB) and subitems may also be utilized to test cognition. While these assessments are often used in subjects that may be suspected of having MCI or Alzheimer's disease, such tests may also be used in healthy subjects long before the development of a cognitive disorder that has been diagnosed by a physician. It is believed that in some dosage forms and in some dosage amounts, the combination of scyllo-inositol and calcifediol may be safely administered to subjects outside of the prescription context and with respect to single active ingredients that have sufficient safety and efficacy data.

### Examples

### Example 1

Clinical Study in a Selected Mild AD MMSE 22-26 Subpopulation and MCI Patient Population with an MMSE of 26-30 Treated with Scyllo-inositol.

Mild AD patients with an MMSE score of 22-26 and MCI patients (MMSE 26-30) with the following baseline characteristics; ADAS≥8 and FAQ≥2 OR/AND ADAS≥8 and CDR≥2 will be selected and combined for enrolment in the study. The selected patients will be divided into two groups; a placebo and scyllo-inositol treated. The patients will be treated with placebo or 250 mg scyllo-inositol BID for a period of up to 18 months. The patients will be followed for safety and efficacy. The key efficacy endpoints (NTB. ADAS-cog 11 and CDR-SB) will be measured at base line and following treatment with placebo or 250 mg BID of scyllo-inositol for a period of 3, 6, 12 and 18 months. Safety will be analyzed at similar time points.

### Example 2

Mouse Study with a Combination of Scyllo-Inositol and Apigenin

### Effect of Scyllo-Inositol and Apigenin Treatment in 5XFAD Transgenic AD Mouse Model.

In order to determine the effects of scyllo-inositol and apigenin alone or in combination on amyloid beta accumulation, number and size of plaques and inflammation in brain, 5XFAD transgenic AD mice are treated daily with different concentrations of scyllo-inositol and apigenin. Eight 5XFAD mice (3 months old) per group are treated with different doses of scyllo-inositol and apigenin daily as described below. The agents dissolve in water as a suspension and are administered by gavage daily. The body weight and clinical appearance of the animals is observed once weekly or more frequently.

Following 3 months of treatment the animals are sacrificed and the brains are removed and one hemisphere is fixed in 4% paraformaldehyde imbedded in paraffin wax. Sections are prepared at 50-um sections and analyzed for amyloid beta 42 and plaques using an anti-amyloid beta antibody and visualized with DAB. Fibrous astrocytes are analyzed with antibodies to GFAP. The amount of amyloid beta staining, number of plaques and size of plaques and number of fibrous astrocytes is quantitated.

The other brain hemisphere is homogenized and prepared for analysis of amyloid beta 40 and 42, and GFAP levels. The level of these proteins in the brain homogenates will be performed by immunoblots and/or radioimmunoassay using antibodies specific to each of the proteins. The groups are as follows:
Group 1 Control—Non-transgenic mouse—parent strain (for control histological staining for amyloid beta levels and GFAP levels, as well as for the preparation of homogenates for quantitation of amyloid beta and GFAP levels)
Group 2 Control—5XFAD mice (untreated) sacrificed at 3 months (baseline for transgenic mice)
Group 3 Control—5XFAD mice (not treated with drug) and sacrifice at 6 months
Group 4 Treated—15 mg/kg of scyllo-inositol (treated for 3 months) and sacrificed at 6 months
Group 5 Treated—40 mg/kg of scyllo-inositol (treated for 3 months) and sacrificed at 6 months
Group 6 Treated—15 mg/kg of apigenin (treated for 3 months) and sacrificed at 6 months
Group 7 Treated—40 mg/kg of apigenin (treated for 3 months) and sacrificed at 6 months
Group 8 Treated—15 mg/kg of scyllo-inositol+15 mg/kg of apigenin (treated for 3 months) and sacrificed at 6 months
Group 9 Treated—15 mg/kg of scyllo-inositol+40 mg/kg of apigenin treated for 3 months) and sacrificed at 6 months
Group 10 Treated—40 mg/kg of scyllo-inositol+15 mg/kg of apigenin treated for 3 months) and sacrificed at 6 months
Group 11 Treated—40 mg/kg of scyllo-inositol+40 mg/kg of apigenin treated for 3 months) and sacrificed at 6 months

It has been previously shown that 10 mg/kg and 30 mg/kg of scyllo-inositol is capable reducing amyloid beta 42 in the brain by 20-30% and the number of plaques by 25-50% (Nature of Medicine publication). These changes were sufficient to improve memory and cognition in a transgenic AD model. Apigenin has been show to increase clearance of amyloid beta by 30-40% in the brain, as well as reduce amyloid beta 42 burden in a transgenic mouse model by 20-30%. Because the mechanism of action of the two active components are so different, there is no way, a priori, to predict whether the combination will lead to any effects or additive effects that are more than, for example, treatment using either drug alone. The studies are therefore designed to, in fact, determine if the addition of apigenin to a course of scyllo-inositol decreases amyloid beta and the number of plaques in the brain of mice in the AD mouse model more than either drug administered alone.

If the administration of the drugs resulted in the maximal known effect for each drug without any confounding factors, one could potentially see a statistically significant decrease of amyloid beta in the brain by an additional 10-20% after treatment with the combination product versus such decrease with either drug alone. The overall decrease in amyloid beta after treatment with the combination of scyllo-inositol and apigenin could be in the range of 30-50% depending on the doses of both drugs. As for the number of plaques, a decrease of 35-70 in the number of plaques with the treatment of scyllo-inositol and apigenin combination can occur.

In clinical trials and/or in products sold as nutraceuticals in humans or animals, the preferred dosage forms include capsules or tablets having both scyllo-inositol and apigenin in varying dosage amounts. These amounts can include various ratios of scyllo-inositol by weight, molarity or as dosed in mgs/kg to the drug apigenin. The combination product may also be administered as separate dosage forms having each active ingredient which are administered at the same time in once a day or twice a day formulations. In some embodiments, the tablets may be in the form of a tablet having an inner core of scyllo-inositol and an outer layer of apigenin mixed with pharmaceutically acceptable tablet excipients such as lubricants or fillers. The tablets may additionally or optionally be enteric coated with, for example, Eudragit US or other suitable enteric coating polymer. The outer layer of apigenin can rapidly dissolve and subsequently increase the expression of the aquaporin 4 channels while the inner core of scyllo-inositol is later released to enter into the interstitial fluid in the brain to disrupt and/or stop the formation of beta amyloid plaques and facilitate the clearance of the fibrils through the expressed aquaporin channels and through the other catabolic paths.

### Example 3

Clinical Study with Scyllo-Inositol and Apigenin

### Patient Population:

Mild AD patients with MMSE scores of 22 to 26 alone or combined with MCI patients with MMSE scores of 27-28 and with predicted more rapid decline in cognition and function based on specific inclusion criteria described below:
1. Mild AD patients with MMSE scores of 22 to 26
2. MCI patients with MMSE scores of 27 to 28 and the following criteria:
   ADAS≥8, FAQ≥2, CDR≥2 or
   ADAS≥7 FAQ≥1 and combined ADAS and FAQ scores must be a ≥13
3. Positive for amyloid beta in the brain method
4. Patients stratified based on MMSE scores for mild AD, Apo e4 genotype, MCI patients

### Treatment Arms for the Study:

Arm #1: Placebo (patients treated with exactly the same conditions as the treated patients below but without active drug—placebo tablet or capsule) Arm #2. Patients will be treated with a tablet or capsule containing 75 mg of scyllo-inositol and 75 mg of apigenin daily for 6 months. The patients will continue treatment in an open label extension and followed for up to an additional 12 months. The patients will be analyzed for efficacy using specific memory tests, and NTB test and its sub-items at 3 and 6 months, and in the OLE study at 12 and 18 months. Safety will be monitored at the same time points as used for efficacy above.

Arm #3: Patients will be treated with a tablet or capsule containing 225 mg of scyllo-inositol and ISO mg of apigenin daily for 6 months. The patients will continue treatment in an open label extension and followed for up to and additional 12 months. The patients will be analysed for efficacy using specific memory tests, and NTB test and its subitems at 3 and 6 months, and in the OLE study at 12 and 18 months. Safety will be monitored at the same time points as used for efficacy above.

### Number of Patients Per Group: 210 Patients

1) Placebo—70
2) Treated arm #1: Scyllo-inositol (75 mg) and apigenin (75 mg)—70
3) Treated arm #2: Scyllo-inositol (225 mg) and apigenin (150 mg)—70

Period of treatment: Patients will be treated for 6 months, followed by up to another 12 months in the open label extension. A blinded and independent group will perform an efficacy and safety analysis analysis at 3 and 6 months for evaluation in the main study. Similarly, the efficacy and safety will be analysed at 12 and 18 months in the open label extension

### Primary and Key End Points for Analysis:

Memory tests will be the primary endpoint, including the NTB test and individual sub items. The specific end points, and potentially other memory and cognition end points may be examined depending on the patient population.

### Safety and Further Analysis:

Typical safety parameters required for the Mild AD and MCI indication will be examined. Results of the Study will show a clinical benefit in subjects receiving the combination product containing scyllo-inositol and apigenin at different doses relative to the placebo group. The data is designed to detect significant changes in specific memory tests and potentially the NTB test.

The preclinical animal studies and clinical studies on scyllo-inositol and apigenin show that the combination is useful for treating and/or to provide to subjects reduce the aggregation of beta amyloid fibrils. It is believed that early intervention and provision of such a combination will enhance cognitive health in such subjects, particularly those subjects approaching fifty years of age or older and which have not yet exhibited signs of cognitive dysfunction which includes memory loss, cognitive impairment of the earliest or continuing formation of aggregates of beta amyloid. The combination of disruption of fibrils and clearance of fibrils along with the additional and significant properties of flavones, particularly apigenin, on inflammation and on aquaporin 4 expression provide enhanced and unexpected properties which are demonstrated in the mice studies and in the clinical trials on subjects which will be treated in the study disclosed herein.

1. A combination of (i) a first compound selected from an inositol or a pharmaceutically acceptable isomer or salt thereof and (ii) a second compound selected from a molecule that upregulates aquaporin 4.

2. The combination according to claim 1 wherein the first compound is selected from scyllo-inositol or other active 1,2,3,4,5,6-cyclohexanehexol and the second compound is selected from the group consisting of molecule flavone.

3. The combination according to claim 2 wherein the first compound is selected from scyllo-inositol and the second compound is selected from apigenin.

4-5. (canceled)

6. A dosage form comprising a combination of (i) a first compound selected from an inositol or a pharmaceutically acceptable isomer or salt thereof and (ii) a second compound selected from a flavone.

7. The dosage form according to claim 6 wherein the first compound is selected from scyllo-inositol or other active 1,2,3,4,5,6-cyclohexanehexol and the second compound is selected from apigenin.

8. The dosage form according to claim 7 wherein the first compound is selected from scyllo-inositol.

9. The dosage form according to claim 8 wherein in the form of a solid, oral dosage form.

10. The dosage form according to claim 9 wherein the dose of scyllo-inositol is about 75 to 150 mgs once or twice daily (BID) and the dose of apigenin is about 60-150 mgs per day.

11. The dosage form according to claim 6 wherein said dosage form is in the form of a capsule or tablet.

12. The dosage form according to claim 11 in the form of a tablet comprising an immediate release formulation of scyllo-inositol and an immediate release formulation of apigenin.

13. The dosage form according to claim 11 in the form of a tablet comprising an immediate release formulation of scyllo-inositol and an extended release formulation of apigenin.

14. A method of treating a subject having an MMSE score in the range of 22-26 with a pharmaceutically effective amount of scyllo-inositol.

15. The method according to claim 14 wherein the subject has Alzheimer's disease.

16. The method according to claim 14 wherein the pharmaceutically effective amount of scyllo-inositol is about 250 mgs BID per day.

17-30. (canceled)

31. A method of supporting cognitive health comprising the administration of a nutraceutically effective amount of a composition comprising scyllo-inositol and apigenin.

32. The method according to claim 31 comprising providing the composition comprising scyllo-inositol and apigenin to subjects over the age of fifty at risk of accumulation of amyloid beta.

33. The method according to claim 31 wherein the subjects exhibit early signs of memory loss.

34. (canceled)
